(19) United States
(12) Patent Application Publication
(10) Pub. No.: US 2008/0302367 A1
(43) Pub. Date: Dec. 11, 2008

(54) Oradent

(76) Inventors: Kameka Walker, Kingston (JM); Gary Bullard, Miramar, FL (US)

Correspondence Address: Gary Bullard, 3804 SW 165th Ave, Miramar, FL, 33027, US

(21) Appl. No.: 11/688,823

(22) Filed: Jun. 7, 2007

(51) Int. Cl. A61F 6/06

(52) U.S. Cl. 128/830

(57) ABSTRACT

The oradent is made of latex or polyurethane material; it is similar to a really small female panty, which covers the entire genital and anus area. It is used in the same manner as dental dams and condoms, to stop the exchange of body fluids during sexual and oral intercourse which will prevent unwanted pregnancies, HIV and STD'S. There are two version of the oradent. One for oral sex only and the other for both oral and penetration sex.

Both versions have an opening (bands) on each side which the legs go through, then it must be pulled up close to the crotch area. The crotch areas have an array of flavors and sizes. The latex sheath is flexible in the vagina and anus areas for slight penetration with tongue during oral sex.

The penetration and oral version has an opening in the crotch with a sack located over the opening of the vagina to be inserted with fingers or with the insertion of the penis. The opening (bands) on each sides of the oradent is designed to stay firmly in place during sexual intercourse. The oradent must be discarded after use and not used twice or flushed.

### BACKGROUND OF THE INVENTION

The Oradent is a revised and upscaled version of the dental dam and condom used to prevent transmission and spread of microorganisms, STDs, pregnancy and body fluids. Prior industry products do not protect the genital and anal areas at the same time as the Oradent. There are not many versions of the dental dam and those versions have to be held in place.

Generally condoms and dental dams are produced from latex, however an estimated Seven percent of the general populations are allergic to latex. In addition the Oradent Could be produced from heat stamped plastic such as polyurethane.

The Oradent would provide protection from sexually transmitted diseases by simply putting on the oradent before engaging in oral sex as well as the standard version will also protect during oral and penetration.

### BRIEF SUMMARY OF INVENTION

The concept of “ORADENT” is to provide protection during oral sex, anal sex, and vaginal intercourse. This product will promote safe sex by preventing the spread of sexually transmitted deceases (STDS), Including human papillomavirus (HPV) while also preventing unwanted pregnancies by blocking the exchange of bodily fluids during sexual intercourse. There are two versions of the Oradent (one for oral sex only and one for penetration and oral) in various sizes and flavors

### BRIEF DESCRIPTION OF THE SEVERAL VIEWS OF THE DRAWING

Accompanying figures and reference alphabets

FIG. 1. Shows full view of Oradent panty version (oral only)

FIG. 2. Shows actual fit of Oradent panty version as depicted in FIG. 1.

FIG. 3. Shows full view of Oradent panty version (penetration and oral)

FIG. 3a shows full view of Oradent panty version (insert sack extended)

FIG. 4. Shows actual fit of Oradent panty version as depicted in FIG. 3.

FIG. 5. Shows the actual fit of the Oradent panty for oral and penetration.

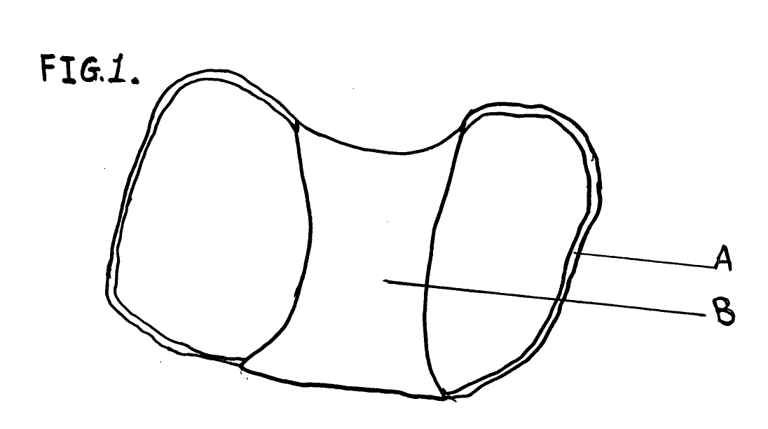

FIG. 1A. Shows band to go around top part of thigh (oral version only)

FIG. 1.B Shows latex area that covers genitals and anus with flexible stretching in both areas.

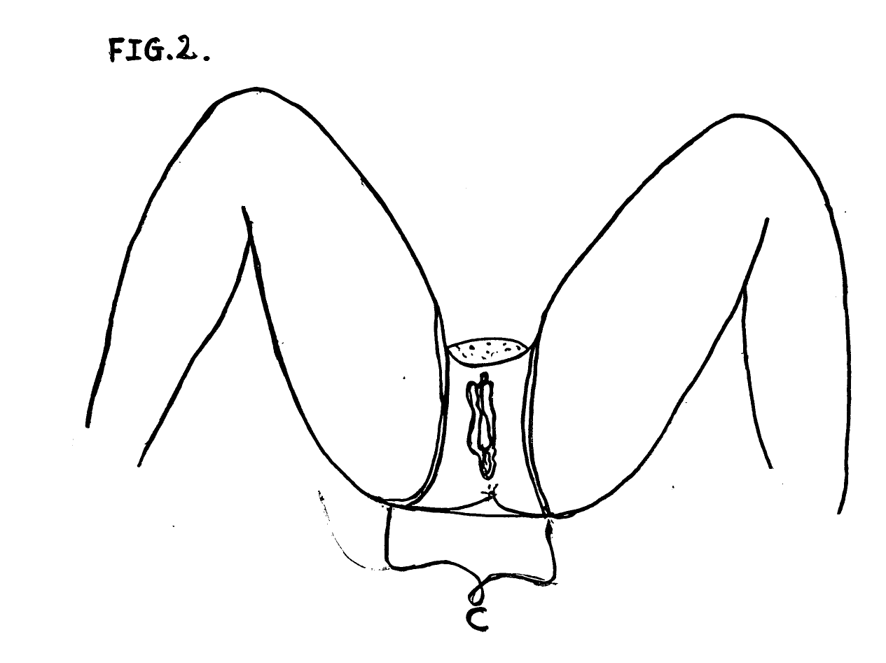

FIG. 2.C. Shows actual position of the band around the top of the thigh

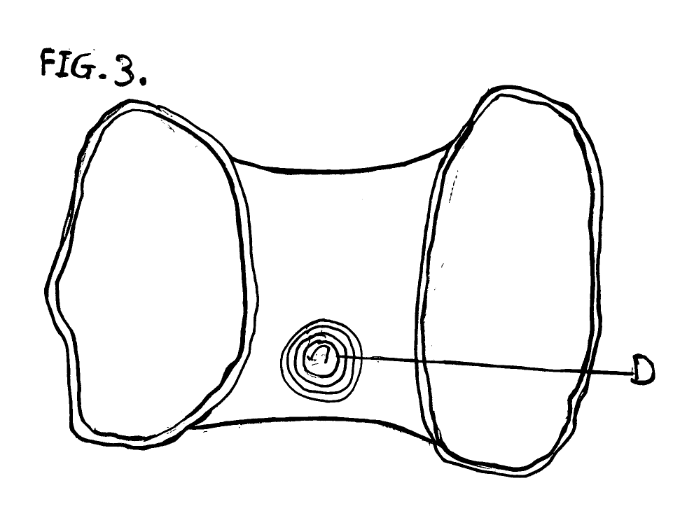

FIG. 3.D. Shows view of vaginal insert sack before extended inwards

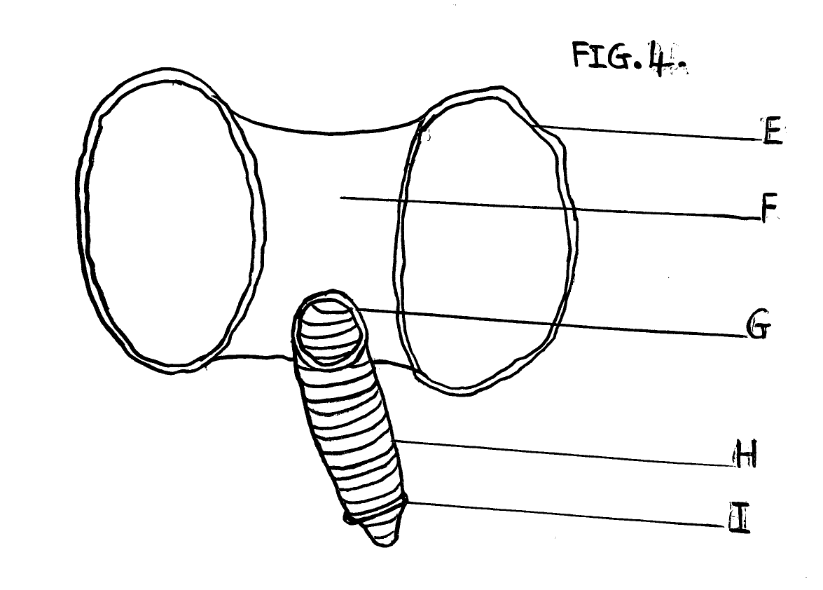

FIG. 4.E. Shows band to go around top part of thigh (oral and penetration version)

FIG. 4.F. Shows latex area that covers genitals and anus with flexible stretching in both areas

FIG. 4.G Shows soft ring that is outside of vagina

FIG. 4.H. Shows latex Insert sack extended

FIG. 4.I Shows soft flexible ring to hold when inserting sack within vagina.

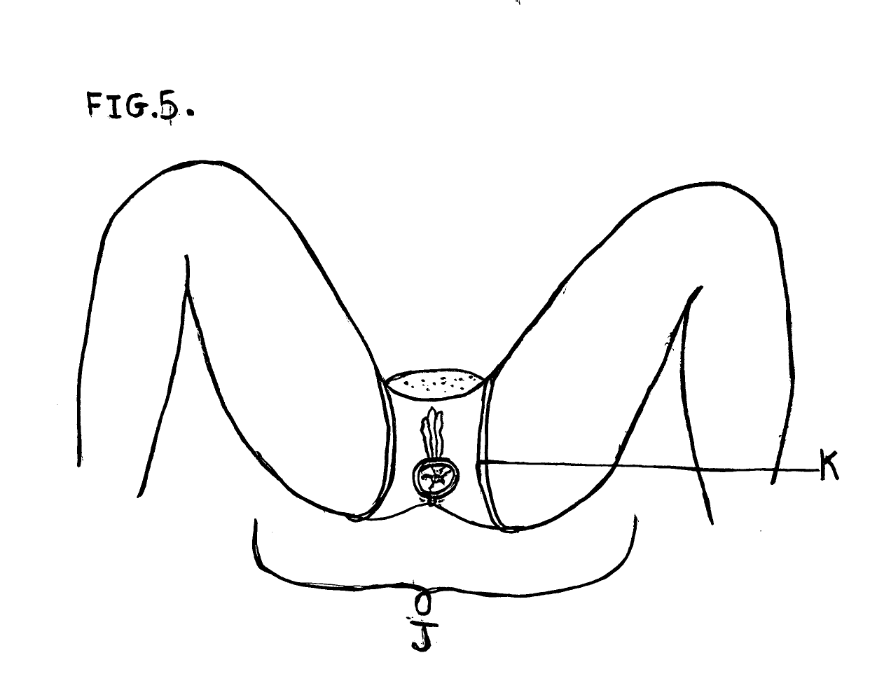

FIG. 5.K. Shows actual position of the band around the top of the thigh and the position of the extended sack when in the vagina

FIG. 5.J Shows the front view and sides of the middle latex section to be slightly concaved for comfort and easy fit to the groin.

### DETAILED DESCRIPTION OF THE INVENTION

The Oradent is a latex rubber modified of a dental dam together with the attributes of a female condom. The Oradent consist of a protective covering of the entire genitalia and anus designed similar to a really small female panties as shown in FIG. 1. It has bands around the opening for the legs that would ensure it would remain securely in place-see FIG. 1A. For one version of the Oradent the crotch area has a latex sheath that covers the vagina and anus as shown in FIG. 1B. FIG. 2 is an actual fit for first version as depicted in FIG. 1.

The second version of Oradent has the insertable sack that would be positioned with-in the vagina as shown in FIG. 3. A soft, flexible ring would rest at the base of the extension for support when there is penetration as shown in FIG. 4G. A second ring at the end of the sack to hold when inserting sack with-in vaginal as shown in FIG. 4I. The area which covers the vagina and anus (FIG. 4F) would contain a wide array of flavors, a wide range of sizes and a variety of colors including transparent. FIG. 5. Shows the actual fit of the oral and penetration version of the oradent.

(FIG. 5K) shows the bands how they will be positioned around the top of the thighs keeping the oradent in place as well as showing the front view of the extended sack covering the vagina. FIG. 5.J shows the sides of the middle section to be slightly concaved for comfort and easy fit to the groin. Standard versions will measure about 7 to 9 inches long by 4 to 6 inches wide.

When properly positioned, the oradent will fully cover the the genitalia area including the anus. There is also a version with out the sack or extension which is mainly for oral sex only.

Both versions will have water based lubricant for (oral version) and spermicidal for (oral and penetration version).

The Oradent will be employed prior to every sexual encounter. After each use Oradent must be discarded and not used twice.

This Product in intended for every and anyone who wants to practice safe sex.

What is claimed is:

1. A female contraceptive that is shaped like a really small panty with a latex concaved sheath which fits over the vagina and anus with bands to place around the thighs to hold firmly in place without the use of hands and protects against the exchange of body fluids, pregnancy and STI'S.

2. The oradent of claim 1 is flexible in middle section area covering the vagina and anus areas for oral only sex.

3. The oradent of claim 1 states wherein the middle section which covers the anus and vagina will have an array of flavors and sizes.

4. The oradent of claim 1 wherein the bands which should be placed around the thigh is attached to the top right end and the bottom right end of middle section, the left band which should be placed round the left leg is attached to the top left end and the bottom left of the middle section.

5. The oradent of claim 1 wherein one type of Oradent will be latex

6. The oradent of claim 1 wherein one type of oradent is heat-stamped plastic or polyurethane.

7. The oradent of claim 1 has water based lubrication.

8. The oradent of claim 1 wherein second version has an insertable sack at opening of vagina area for penis insertion which is lubricated with spermicide.

9. The Oradent of claim 8 has a soft, flexible ring would rest at the base of the extension for support when there is penetration

10. The oradent of claim 8 has a second ring at end of the sack to hold when inserting in the vagina.

11. The oradent of claim 8 states wherein the middle section which covers the anus and vagina will have an array of flavors and sizes.

12. The oradent of claim 8 wherein the bands which should be placed around the thigh is attached to the top right end and the bottom right end of middle section, the left band which should be placed round the left leg is attached to the top left end and the bottom left of the middle section.

13. The oradent of claim 8 wherein one type of Oradent will be latex.

14. The oradent of claim 8 wherein one type of oradent is heat-stamped plastic or polyurethane.
